(19) United States
(12) Patent Application Publication
(10) Pub. No.: US 2001/0046674 A1
(43) Pub. Date: Nov. 29, 2001

(54) Signaling aptamers that transduce molecular recognition to a differential signal

(76) Inventor: Andrew Ellington, Austin, TX (US)

Correspondence Address: Benjamin Aaron Adler, ADLER & ASSOCIATES, 8011 Candle Lane, Houston, TX, 77071, US

(21) Appl. No.: 09/776,252

(22) Filed: Feb. 2, 2001

Related U.S. Application Data
(60) Provisional application No. 60/179,913, filed on Feb. 3, 2000.

(51) Int. Cl. C12Q 1/68

(52) U.S. Cl. 435/6

(57) ABSTRACT

The present invention provides a method of transducing the conformational change undergone by a signaling aptamer upon binding a ligand to a differential signal generated by a reporter molecule. Also provided is a method of detecting and quantitating a ligand in solution using an aptamer conjugated to a fluorescent dye (signaling aptamer) to bind to the ligand and measuring the resultant optical signal generated.

### CROSS-REFERENCE TO RELATED APPLICATION This non-provisional patent application claims benefit of provisional patent application U.S. serial No. 60/179,913, filed Feb. 3, 2000, now abandoned.

### BACKGROUND OF THE INVENTION

1. Field of the Invention

The present invention relates generally to the fields of biochemistry and biophysics. More specifically, the present invention relates to nucleic acid binding species or aptamers containing reporter molecules used to signal the presence of cognate ligands in solution.

2. Description of the Related Art

The SELEX method (hereinafter termed SELEX), described in U.S. Pat. No. 5,475,096 and U.S. Pat. No. 5,270,163 provides a class of products which are nucleic acid molecules, each having a unique sequence, each of which has the property of binding specifically to a desired target compound or molecule. Each nucleic acid molecule is a specific ligand of a given target compound or molecule. SELEX is based on the unique insight that nucleic acids have sufficient capacity for forming a variety of two- and three-dimensional structures and sufficient chemical versatility available within their monomers to act as ligands (form specific binding pairs) with virtually any chemical compound, whether monomeric or polymeric. Molecules of any size can serve as targets.

The SELEX method involves selection from a mixture of candidates and step-wise iterations of structural improvement, using the same general selection theme, to achieve virtually any desired criterion of binding affinity and selectivity. Starting from a mixture of nucleic acids, preferably comprising a segment of randomized sequence, the method includes steps of contacting the mixture with the target under conditions favorable for binding, partitioning unbound nucleic acids from those nucleic acids which have bound to target molecules, dissociating the nucleic acid-target pairs, amplifying the nucleic acids dissociated from the nucleic acid-target pairs to yield a ligand-enriched mixture of nucleic acids, then reiterating the steps of binding, partitioning, dissociating and amplifying through as many cycles as desired.

Within a nucleic acid-mixture containing a large number of possible sequences and structures there is a wide range of binding affinities for a given target. A nucleic acid mixture comprising, for example a 20 nucleotide randomized segment can have 4sup.20 candidate possibilities. Those which have the higher affinity constants for the target are most likely to bind to the target. After partitioning, dissociation and amplification, a second nucleic acid mixture is generated, enriched for the higher binding affinity candidates. Additional rounds of selection progressively favor the best ligands until the resulting nucleic acid mixture is predominantly composed of only one or a few sequences. These can then be cloned, sequenced and individually tested for binding affinity as pure ligands.

Cycles of selection, partition and amplification are repeated until a desired goal is achieved. In the most general case, selection/partition/amplification is continued until no significant improvement in binding strength is achieved on repetition of the cycle. The method may be used to sample as many as about 10.sup.18 different nucleic acid species. The nucleic acids of the test mixture preferably include a randomized sequence portion as well as conserved sequences necessary for efficient amplification. Nucleic acid sequence variants can be produced in a number of ways including synthesis of randomized nucleic acid sequences and size selection from randomly cleaved cellular nucleic acids. The variable sequence portion may contain fully or partially random sequence; it may also contain subportions of conserved sequence incorporated with randomized sequence. Sequence variation in test nucleic acids can be introduced or increased by mutagenesis before or during the selection/partition/amplification iterations.

Most conventional diagnostic assays rely on the immobilization of either biopolymer receptors or their ligands. Such assays tend to be time-consuming and labor-intensive, necessitating the development of homogenous assay formats that do not require multiple immobilization or washing steps. Aptamers have been introduced previously into diagnostic assays, although their primary use is as substitutes for antibodies. For example, Gilardi et. al. have conjugated fluorescent dyes to maltose-binding protein and were able to directly read maltose concentrations in solution¹, and Marvin and Hellinga have conjugated fluorescent dyes to glucose-binding protein and followed glucose concentrations in solution².

Oligonucleotides and nucleic acids have previously been adapted to sense hybridization³ and could potentially be used to detect metals.⁴ Aptamers have been selected against a wide array of target analytes, e.g., ions, small organics, proteins, and supramolecular structures such as viruses or tissues^{18,19}.

The conversion of ligand-binding proteins⁵ or small molecules⁶ to biosensors is highly dependent on the structure and dynamics of a given receptor, thus, it may be simpler to convert aptamers to biosensors.⁷⁻⁸ Aptamers generally undergo an ‘induced fit’ conformational change in the presence of their cognate ligands,⁹ and thus an appended dye easily undergoes a ligand-dependent change in its local environment. In contrast to other reagents, e.g., antibodies, aptamers are readily synthesized and dyes are introduced easily into specific sites. Thus, aptamer biosensors can be quickly generated using both rational and random engineering strategies.

The prior art is deficient in the lack of nucleic acid binding species (aptamers) containing reporter molecules that signal the presence of cognate ligands in solution. The present invention fulfills this long-standing need and desire in the art.

### SUMMARY OF THE INVENTION

In one embodiment of the present invention there is provided a method of transducing the conformational change of a signaling aptamer upon binding a ligand to a differential signal generated by a reporter molecule comprising the steps of contacting the signaling aptamer with the ligand wherein the signaling aptamer binds the ligand; and detecting the differential signal generated by the reporter molecule resulting from the conformational change of the signaling aptamer upon binding the ligand thereby transducing the conformational change.

In another embodiment of the present invention there is provided a method of transducing the conformational change of a signaling aptamer upon binding a ligand to an optical signal generated by a fluorescent dye. This method comprises the steps of contacting the signaling aptamer with the ligand wherein the signaling aptamer binds the ligand; and detecting the optical signal generated by the fluorescent dye resulting from the conformational change of the signaling aptamer upon binding the ligand thereby transducing the conformational change.

In yet another embodiment of the present invention there is provided a method for quantitating the ligand disclosed supra comprising the steps of contacting the signaling aptamer disclosed supra with the ligand wherein the signaling aptamer binds the ligand; and measuring the increase in the optical signal disclosed supra resulting from the signaling aptamer binding the ligand; wherein the increase in the optical signal positively correlates with the quantity of ligand bound to the signaling aptamer.

Other and further aspects, features, benefits, and advantages of the present invention will be apparent from the following description of the presently preferred embodiments of the invention given for the purpose of disclosure.

### BRIEF DESCRIPTION OF THE DRAWINGS

So that the matter in which the above-recited features, advantages and objects of the invention, as well as others which will become clear, and are attained and can be understood in detail, more particular descriptions of the invention are briefly summarized above may be had by reference to certain embodiments thereof which are illustrated in the appended drawings. These drawings form a part of the specification. It is to be noted, however, that the appended drawings illustrate preferred embodiments of the invention and therefore are not to be considered limiting in their scope.

FIG. 1 shows the three-dimensional models of anti-adenosine aptamers derived from NMR analysis.¹¹⁻¹² Some of the sites chosen for dye incorporation into either RNA, ATP-R-Ac13 (blue), or DNA, DFL7-8 (orange), aptamers are shown in yellow. Bound adenosines are shown in purple.

FIG. 2 shows the sites of dye incorporation into RNA and DNA aptamers. In FIG. 2A in the RNA aptamers acridine is incorporated in place of residue 13 (ATP-R-Ac13). Fluorescein is incorporated at the 5′ end (ATP-R-F1), at the 5′ end with a heptaadenyl linker (ATP-R-F2), and in place of residue 13 (ATP-R-F13). In FIG. 2B in the DNA aptamers. fluorescein was incorporated at the 5′ end (DFL0), in place of residue 7 (DFL7), and in between residues 7 and 8 (DFL7-8). Residues are numbered from the 5′ end on the secondary structures.

FIG. 3 shows the specificities of the signaling aptamers ATP-R-Ac13 (FIG. 3A) and DFL7-8 (FIG. 3B). The fractional increase in relative fluorescence units (ARFU) was measured in the presence of ATP, GTP, CTP, and UTP (1 mM ligand for ATP-R-Ac13, 200 μM ligand for DFL7-8).

FIG. 4 shows the mutant versions of signaling aptamers ATP-R-Ac13 (FIG. 4A) and DFL7-8 (FIG. 4B) do not signal. The ΔRFU was measured in the presence of ATP (1 mM ligand for ATP-R-Ac13 and Mut34, 250 μM ligand for DFL7-8 and Mut9/22).

FIG. 5 shows the response curves for the signaling aptamers ATP-R-Ac13 (FIG. 5A) and DFL7-8 (FIG. 5B). The ΔRFU plotted at various concentrations of ATP (●) and GTP (▪). Data points are shown as an average of three values with standard deviations. Data was curve-fitted using the program Kaleidograph (Synergy Software).

FIG. 6 shows the Scatchard plot derived from the response curve of the DNA signaling aptamer. The fractional increase in RFU, ΔRFU (x axis), is plotted against the ratio of ΔRFU/[ATP] (γ axis).

FIG. 7 shows the elution profiles for the signaling aptamer DFL7-8 (FIG. 7A) and its double mutant Mut9/22 (FIG. 7B). After applying the radiolabled aptamer, the column was washed with 44 ml of selection buffer. A 0.3 mM GTP solution in selection buffer (15 ml) was applied (first arrow from left). After washing the column with an additional 10 ml of selection buffer (second arrow), a 0.3 mM ATP solution in selection buffer (15 ml) was added (third arrow).

### DETAILED DESCRIPTION OF THE INVENTION

In one embodiment, the present invention is directed to a method of transducing the conformational change of a signaling aptamer upon binding a ligand to a differential signal generated by a reporter molecule comprising the steps of contacting the signaling aptamer with the ligand wherein the signaling aptamer binds the ligand; and detecting the differential signal generated by the reporter molecule resulting from the conformational change of the signaling aptamer upon binding the ligand thereby transducing the conformational change.

The differential signal can be optical, electrochemical or enzymatic. Representative examples of optical signals are fluorescence, colorimetric intensity, anisotropy, polarization, lifetime, emission wavelength, and excitation wavelength. The reporter molecule generating these signals can be covalently bound to the aptamer during chemical synthesis, during transcription or post-transcriptionally or may be appended to the aptamer non-covalently. The reporter molecule can be a fluorescent dye such as acridine or fluorescein. The aptamer may be optionally modified DNA or RNA, but may not comprise a protein or a biopolymer; the ligand may be a non-nucleic acid molecule bound by the signaling aptamer. The ligand and the signaling aptamer may be in solution. Additionally, the signaling aptamer may be immobilized on a solid support and, furthermore, may be immobilized on the solid support in parallel to form signaling chips.

In another embodiment of the present invention there is provided a method of transducing the conformational change of a signaling aptamer upon binding a ligand to an optical signal generated by a fluorescent dye comprising the steps contacting the signaling aptamer with the ligand wherein the signaling aptamer binds the ligand; and detecting the optical signal generated by the fluorescent dye resulting from the conformational change of the signaling aptamer upon binding the ligand thereby transducing the conformational change.

In this aspect of the present invention the optical signals may be as disclosed herein. The reporter molecule may be a fluorescent dye such as acridine or fluorescein. It is covalently bound to the aptamer either replacing a nucleic acid in the aptamer or inserted between two nucleic acids without interfering with the ligand binding site. The aptamer may be an anti-adenosine RNA aptamer such as ATP-R-Ac13 or an anti-DNA aptamer such as DFL7-8. In such cases the ligand is adenosine. The ligand and signaling aptamer may be in solution or the signaling aptamer may be immobilized on a solid support. Signaling chips may be formed by immobilizing the signaling aptamer in parallel.

In yet another embodiment of the present invention there is provided a method for quantitating the ligand disclosed supra comprising the steps of contacting the signaling aptamer disclosed supra with the ligand wherein the signaling aptamer binds the ligand; and measuring the increase in the optical signal disclosed supra resulting from the signaling aptamer binding the ligand; wherein the increase in the optical signal positively correlates with the quantity of ligand bound to the signaling aptamer.

The present invention is directed toward a method of detecting and quantitating the presence of cognate ligands or analytes in solution using engineered aptamers that contain, inter alia, fluorescent dyes.

As used herein, the term “aptamer” or “selected nucleic acid binding species” shall include non-modified or chemically modified RNA or DNA. Inter alia, the method of selection may be by affinity chromatography or filter partitioning and the method of amplification by reverse transcription (RT), polymerase chain reaction (PCR) or isothermal amplification.

As used herein, the term “signaling aptamer” shall include aptamers with reporter molecules appended in such a way that upon conformational changes resulting from the aptamer's interaction with a ligand, the reporter molecules yield a differential signal.

As used herein, the term “reporter molecule” shall include, but is not limited to, dyes that signal via fluorescence or calorimetric intensity, anisotropy, polarization, lifetime, or changes in emission or excitation wavelengths. Reporter molecules may also include molecules that undergo changes in their electrochemical state such as in an oxidation-reduction reaction wherein the local environment of the electron carrier changes the reducing potential of the carried or may include enzymes that generate signals such a s beta-galactosidase or luciferase.

As used herein, the term “ligand” shall include any molecule that binds to the aptamer excepting nucleic acid sequences. Ligands may, however, be nucleic acid structures such as stem-loops.

As used herein, the term “appended” shall include, but is not limited to, covalent coupling, either during the chemical synthesis or transcription of the RNA or post-transcriptionally. May also involve non-covalent associations; e.g., an aptamer non-covalently bound to the active site of an enzyme is released upon interaction with a ligand and activates the enzyme.

As used herein, the term “conformational changes” shall include, but is not limited to, changes in spatial arrangements including subtle changes in chemical environment without a concomitant spatial arrangement.

As used herein, the term “differential signal” shall include, but is not limited to, measurable optical, electrochemical or enzymatic signals.

The following examples are given for the purpose of illustrating various embodiments of the invention and are not meant to limit the present invention in any fashion.

### EXAMPLE 1

### Materials

ATP (disodium salt) and GTP (disodium salt) were purchased from Roche Molecular Biochemicals, and ATP agarose (C8 linkage, 9 atom spacer) was purchased from Sigma. Fluorescein phosphoramidite, 5′-fluorescein phosphoramidite, and acridine phosphoramidite were purchased from Glen Research. T4 polynucleotide kinase and polynucleotide kinase buffer were purchased from New England Biolabs. Radioactive [γ-³²P] ATP was purchased from ICN.

### EXAMPLE 2

### Preparation of Signaling Aptamers

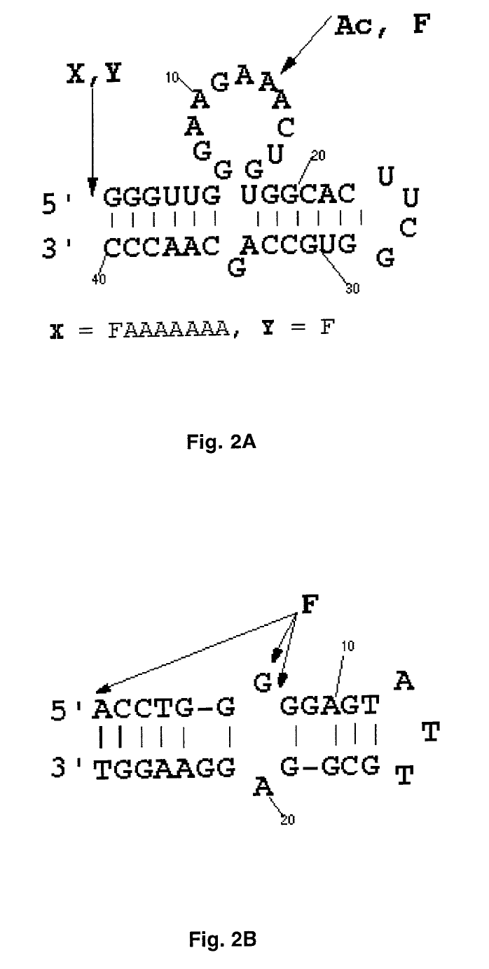

A series of aptamer-dye conjugates (FIG. 2) were synthesized and deprotected as described previously.²⁰⁻²³ Fluorescein phosphoramidite and acridine phosphoramidite were used in the syntheses of the internally-labeled aptamers while the terminally-labeled aptamers are generated using 5′-fluorescein phosphoramidite. Deprotection of the RNA aptamer-dye conjugates was carried out using a procedure modified from Wincott, et al.²³ In the first part of the deprotection, the resins are suspended in 3:1 NH₄OH:EtOH for 13 hours at room temperature, rather than for 17 hours at 55° C. The aptamers are purified by polyacrylamide gel-electrophoresis, eluted with 0.3 M NaOAc overnight at 37° C., and ethanol precipitated. The aptamers were resuspended in 50 μl H₂O and subsequently quantitated by measuring the A₂₆₀ using an extinction coefficient of 0.025 ml cm⁻¹ μg⁻¹ for RNA, and 0.027 ml cm⁻¹ μg⁻¹ for DNA.

The aptamers were thermally equilibrated in selection buffer and conditions were empirically determined to give the optimal fluorescence intensity. Before taking fluorescence measurements, the RNA aptamers (500 nM) were suspended in selection buffer, 300 mM NaCl, 20 mM Tris-HCl, pH 7.6, 5 mM MgCl₂,¹⁶ heat denatured at 65° C. for 3 min, and then slow-cooled to 25° C. in a thermocycler at a rate of 1° C. per 12 seconds. The DNA aptamers (150 nM) were suspended in selection buffer,¹⁷ heat denatured at 75° C. for 3 min, and allowed to cool to room temperature over 10-15 minutes.

### EXAMPLE 3

### Fluorescence Measurements

All fluorescence measurements are taken on a Series 2 Luminescence Spectrometer from SLM-AMINCO Spectronic Instruments. The experimental samples were excited at their respective maximums (acridine λₑₓ=450 nm; fluorescein λₑₓ=495 nm) and fluorescence intensity were measured at the corresponding emission maximums, (acridine, λₑₘ=495 nm; fluorescein, λₑₘ=515 nm). The aptamer solutions (200 μl for RNA, 1,000 μl for DNA) were pipetted into a fluorimeter cell (Starna Cells, Inc.) and ligand solutions of varying concentrations but standard volumes (50 μl for RNA, 1.5 μl for DNA) are added.

### EXAMPLE 4

### Measurements of Binding Affinities by Isochratic Elution

For 5′ end-labeling, the aptamers were incubated for 1 hour at 37° C. in a T4 polynucleotide kinase reaction mix (1 μl T4 polynucleotide kinase (10 units), 2 μl DNA, 0.5 μl 10× polynucleotide kinase buffer, 0.5 μl [γ-³²P] ATP (7000 Ci/mmol), 6 μl H₂O for a total volume of 10 μl). A column of ATP agarose, with a total volume (Vₜ) of 1.5 ml and a void volume (Vₒ) of 1.16 ml was equilibrated with 25 ml selection buffer. Aptamers (10 μg) were thermally equilibrated and applied to the column. The concentration of ATP ([L], see below) on the column is 2.6 mM. The column was then washed with selection buffer and 1 ml fractions are collected. Portions (5 μl) of each fraction were spotted on a nylon filter and the amount of radioactivity present is quantitated with a Phosphorimager (Molecular Dynamics). The column was developed with an additional 44 ml of selection buffer, followed by 15 ml of a 0.3 mM GTP solution in selection buffer. After washing the column with an additional 10 ml of selection buffer, 15 ml of a 0.3 mM ATP solution in selection buffer completely elutes any remaining radioactivity. For the aptamer DFL7-8, a final elution volume (Vₑ) of 73 ml was used to develop the column prior to the addition of the ATP solution. An upper bound for the K_{d} of the signaling aptamer for ATP-agarose is calculated using the equation:

K_{d}=[L]*(Vₜ-Vₒ)/(Vₑ-Vₒ).¹⁶

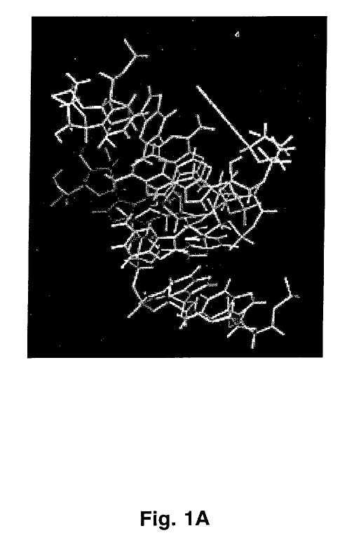

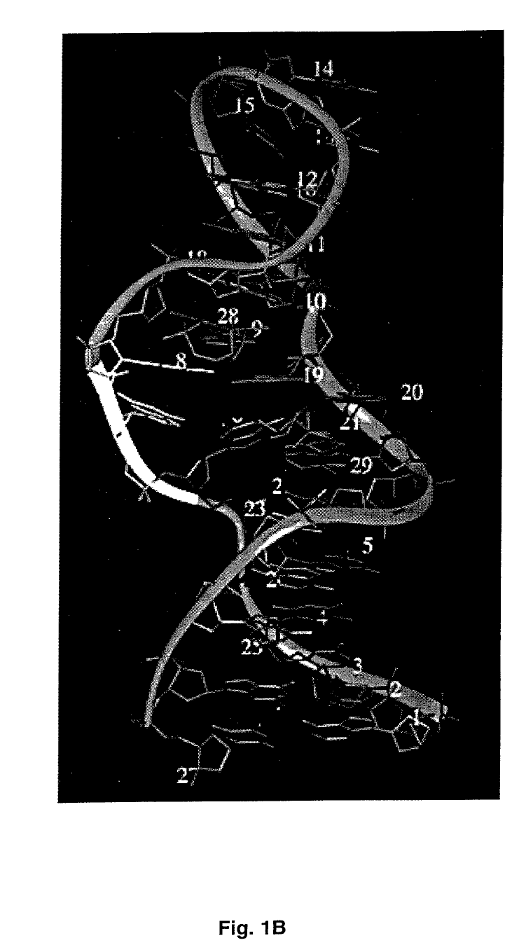

Several three-dimensional structures of aptamers that bind small, organic ligands have been published.¹⁰⁻¹⁴ The structures of two anti-adenosine aptamers^{11,12,15}, one selected from an RNA pool¹⁶ and one selected from a DNA pool,⁷ were used herein for the design of signaling aptamers (FIG. 1). The program Insight 2 (Molecular Simulations) was used to visualize and manipulate the structures of these anti-ATP aptamers. Fluorescent dyes were placed adjacent to functional residues, and the signaling abilities of the resultant chimeras were evaluated by determining whether changes in fluorescence intensity occurred in the presence of the cognate ligand, ATP.

Different anti-adenosine signaling aptamers made from RNA and DNA selectively signal the presence of adenosine in solution. Increases in fluorescence intensity reproducibly follow increases in adenosine concentration, and are used for quantitation. In the methods of the present invention, fluorophores were placed either in proximity to the ligand-binding sites of aptamers, to avoid blocking or disrupting them, or were placed so that larger, ligand-induced conformational changes in aptamer structure (e.g., helical rotation) can be monitored. For example, residue 13 of the anti-adenosine RNA aptamer was adjacent to the binding pocket but does not participate in interactions with ATP; instead the residue points outwards into solution (FIG. 1A). Therefore, an acridine moiety was introduced into the RNA aptamer in place of the adenosine at position 13, ATP-R-Ac13 (FIG. 2). Similarly, residue 7 in the DNA aptamer is in proximity of the binding site, and does not directly interact with ATP (FIG. 1B). Thus, fluorophores replace residue 7 and were inserted between residues 7 and 8, DFL-7 and DFL7-8, respectively (FIG. 2).

Of the various constructs tested, the ATP-R-F1, ATP-R-F2, ATP-R-F13, DFL0, and DFL7 aptamers show an insignificant change in fluorescence intensity (5% or less) upon the addition of ATP. However, the ATP-R-Ac13 and DFL7-8 aptamers showed marked increases in fluorescence intensity in the presence of 1 mM ATP. The increases in response ranged from 25 to 45%.

### EXAMPLE 5

### Specificity Of The Signaling Aptamers

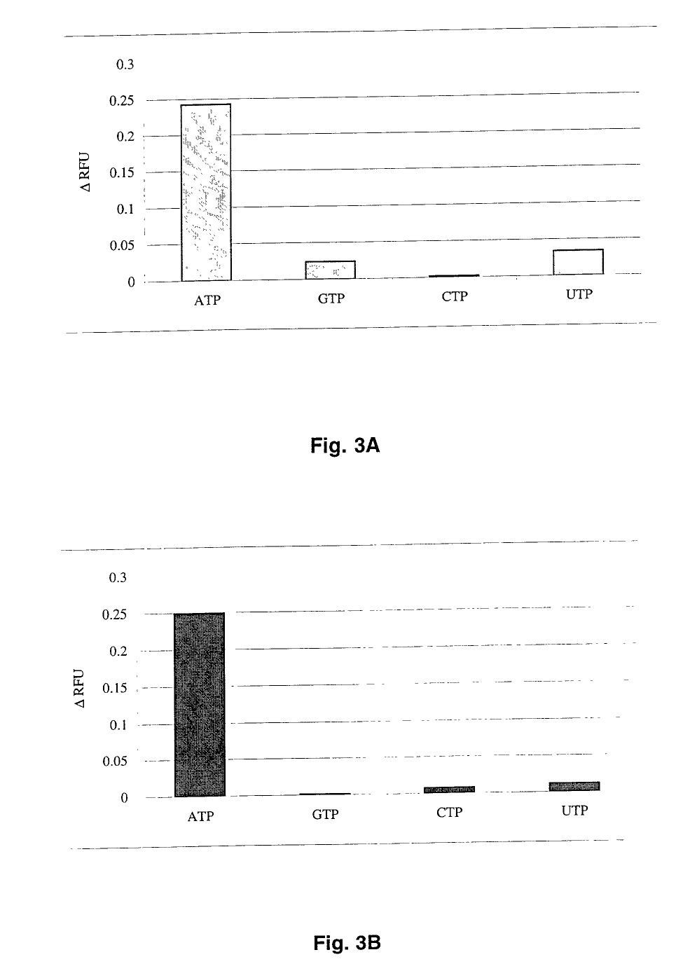

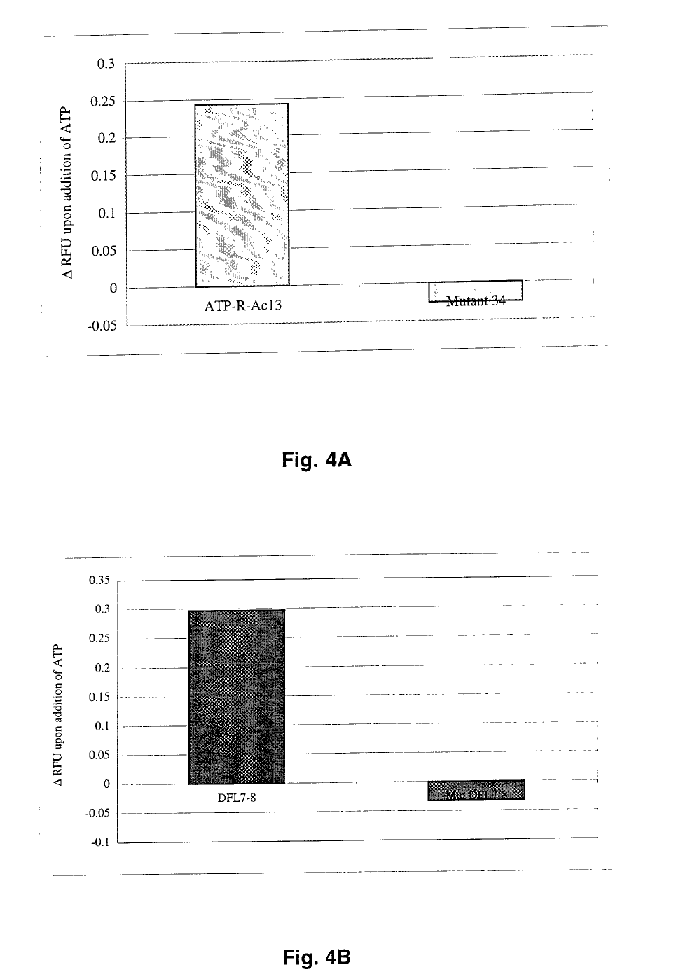

To assess the specificity of the ATP-R-Ac13 (FIG. 3A) and DFL7-8 (FIG. 3B) signaling aptamers for ATP, changes in fluorescence were measured in the presence of GTP, CTP, and UTP. No significant ligand-dependent increases in fluorescence were observed. In addition, mutant versions of ATP-R-Ac13 and DFL7-8 that did not bind to ATP are constructed by omitting or replacing key functional residues. Residue G34 of the RNA aptamer is known from mutagenesis studies to be essential for binding¹⁶, while residues G9 and G22 in the DNA aptamer are critical contacts for the ATP ligands. A mutant of the RNA aptamer lacking G34 (Mut 34) (FIG. 4A) and a double mutant of the DNA aptamer in which both G9 and G22 were replaced with cytidine residues (Mut 9/22) (FIG. 4B) were constructed. The mutant signaling aptamers show no ATP-dependent increases in fluorescence.

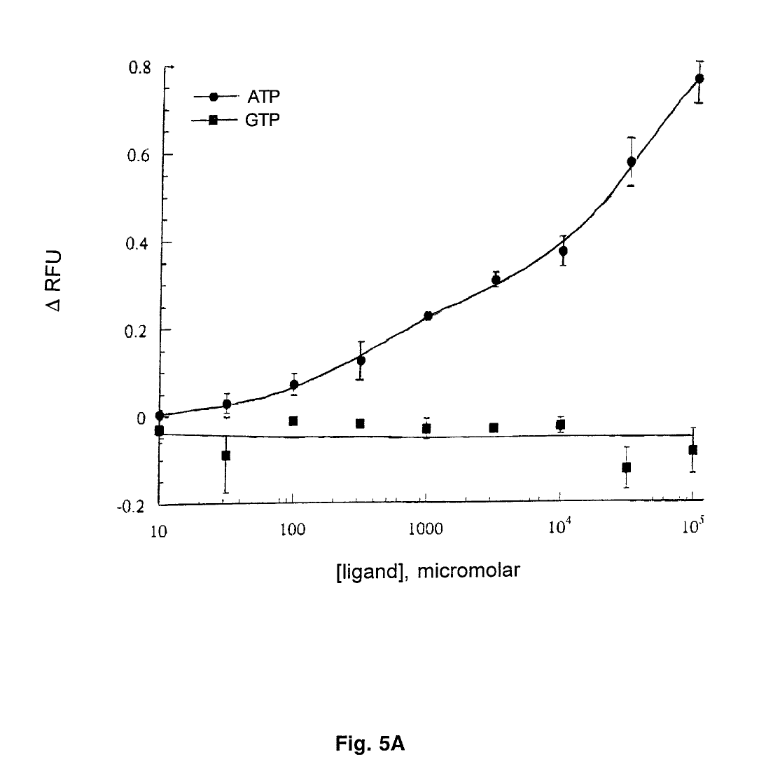

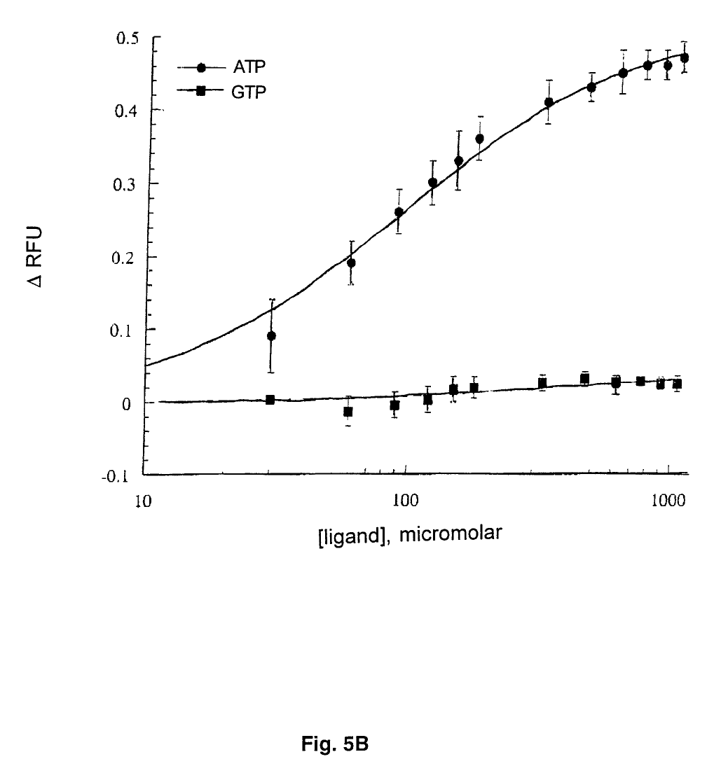

To demonstrate that signaling aptamers can be used to quantitate analytes in solution, response curves are obtained by measuring the fluorescence intensities of ATP-R-Ac13 (FIG. 5A) and DFL7-8 (FIG. 5B) as a function of ATP and GTP concentrations. Both signaling aptamers show a graded increase in fluorescence intensity with ATP, but little or no change in fluorescence intensity with GTP. While the response curves for the signaling aptamers were completely reproducible they could not be fit by simple binding models based on the reported K_{d}'S of the original aptamers. However, the original binding data for the DNA aptamer¹⁷ is based on the assumption that it contained only a single ligand-binding site, while the NMR structure reveals two ligand-binding sites.

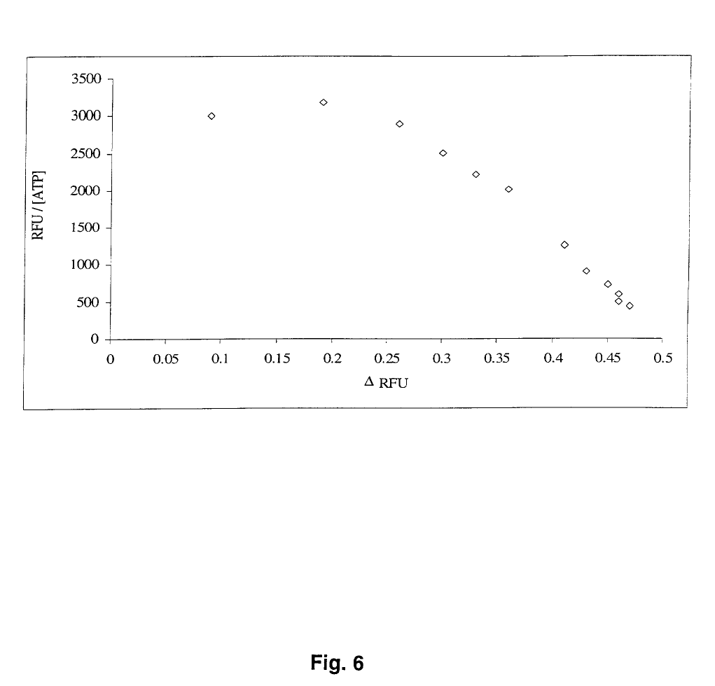

To determine whether the signaling aptamer was detecting both ATP-binding sites, the change in fluorescence was plotted against the ratio of the change in fluorescence to the concentration of unbound ATP. The resulting non-linear Scatchard plot (FIG. 6) is biphasic, suggesting that multiple binding sites are perceived. The signaling data is fit to a model in which the aptamer cooperatively binds to two ATP molecules, using the following equation:

$$( F - {F}_{0} ) = \frac{{K}_{1} ( {F}_{1} - {F}_{0} ) [ L ] + {K}_{1} {{K}_{2} ( {F}_{2} - {F}_{0} ) [ L ]}^{2}}{1 + {K}_{1} [ L ] + {K}_{1} {{K}_{2} [ L ]}^{2}}$$

F: Fluorescent Signal

F₀: Fluorescence of uncomplexed substrate

F₁: Fluorescence of singly bound substrate

F₂: Fluorescence of doubly bound substrate

K₁: Formation constant of first order complex

K₂: Formation constant of second order complex

This analysis yields two dissociation constants, indicating a higher affinity site with a K_{d,1} (1/K₁)of 30 +/−18 μM, and a lower affinity site with a K_{d,2} (1/K₂) of 53 +/−μM. The relative change in fluorescence upon binding first ATP (F₁) was calculated to be negligible, −0.004%, while the relative change in fluorescence due to the formation of the ternary complex (F₂) is calculated to be 49%. The similarity in affinity between the two binding sites is consistent with the sequence and structural symmetry of the DNA, anti-adenosine aptamer. As the greatest change in fluorescence was observed upon ternary complex formation, the affinity of the site containing the fluorescein reporter was perturbed slightly and the signaling aptamer is primarily reporting ligand interactions with this site.

The binding abilities of the signaling aptamers were independently examined using an isocratic elution technique that determines aptamer K_{d}'s for ATP.¹⁶ The signaling aptamers were applied to an ATP affinity column and are eluted progressively with buffer and nucleotides. The RNA signaling aptamer ATP-R-Ac13 bound poorly to the column; its estimated K_{d} is greater than millimolar. These results accord with the relatively large amounts of ATP required to generate a signal (FIG. 5A). The diminution in the affinity of the RNA aptamer upon the introduction of acridine is similar to diminutions in affinity observed upon the introduction of dyes into maltose- and glucose-binding proteins.^{1,2}

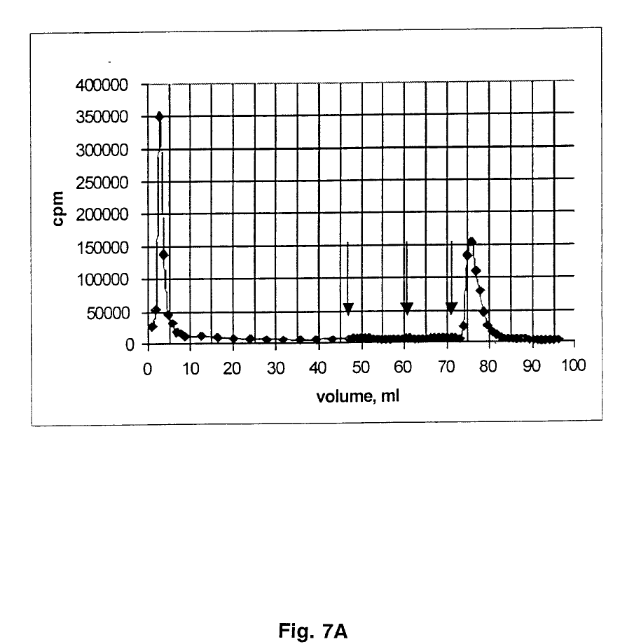

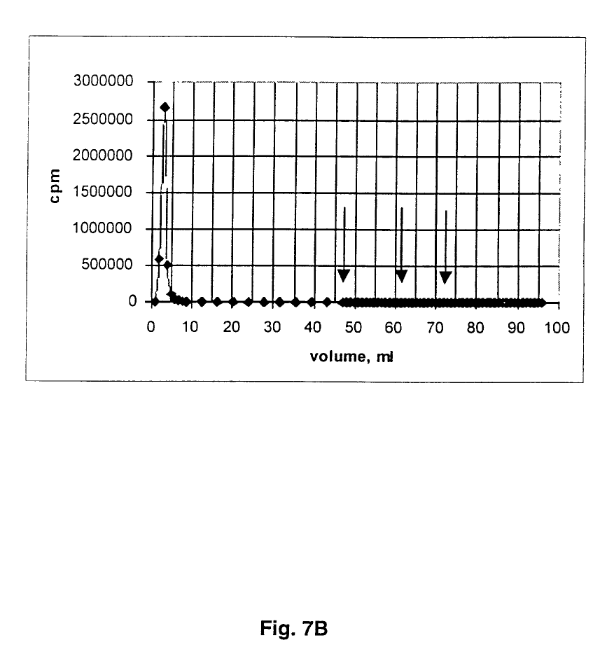

In contrast, the DNA signaling aptamer DFL7-8 (FIG. 7A) has an apparent K_{d} that is lower than 13 micromolar, and can not be eluted from the ATP affinity column with GTP. The affinity of the DNA aptamer inferred from column chromatography is comparable to the calculated affinity of the lower affinity site, above. The non-signaling double mutant, Mut9/22, did not bind to the affinity column (FIG. 7B). The lower K_{d} of the DNA signaling aptamer relative to the RNA signaling aptamer accords with a better signaling response by the DNA signaling aptamer (FIG. 5B). However, it is difficult to directly compare binding and signaling studies with the DNA aptamer, since the unmodified aptamer contains two, cooperative adenosine binding sites¹⁷ which may have been differentially affected by the introduction of the dye.

### EXAMPLE 6

### Other Signaling Aptamers

It is contemplated that reporter molecules comprising a signaling aptamer may be molecules other than fluorescent dyes or other fluors and may generate a differential signal other than optical. Such molecules may undergo changes in their electrochemical state, i.e., a change in redox potential resulting from a change in the local environment of the electron carrier could generate a differential signal. In such interactions, the conformational change may not be spatial, but a change in chemical environment. Alternatively, a reporter molecule could be an enzyme that in itself can generate a differential signal, e.g., beta-galactosidase or luciferase.

As such a reporter molecule may be non-covalently bound to an aptamer. A non-covalent association of the reporter molecule with, for example, the active site of an enzyme could generate a differential signal upon interaction with a ligand; the binding of the ligand to the signaling aptamer alters the non-covalent association of the reporter molecule with the active site and thereby activates the enzyme.

### EXAMPLE 7

### Diagnostic Assays

The fact that aptamer-dye conjugates can directly signal the presence and amount of analytes in solution without the need for prior immobilization or washing steps allows aptamers to be used in ways that are currently unavailable to other aptamers such as antibodies. Numerous new reagents for sensor arrays may be quickly synthesized by the simple addition of fluorescent dyes to extant aptamers, as described herein. The fact that the first generation of designed compounds can detect analytes in the micromolar to millimolar range makes this possibility even more likely. The sensitivity of signaling aptamers is further refined by the incorporation of a wider range of dyes at a wider range of positions.

The following references are cited herein. 1. Gilardi, G.; Zhou, L. Q.; Hibbert, L.; Cass, A. E. Anal Chem 1994, 66, 3840-7. 2. Marvin, J. S.; Corcoran, E. E.; Hattangadi, N. A.; Zhang, J. V.; Gere, S. A.; Hellinga, H. W. Proc Natl Acad Sci U S A 1997, 94, 4366-71. 3. Tyagi, S.; Kramer, F. R. Nat Biotechnol 1996, 14, 303-8. 4. Walter, F.; Murchie, A. ; Lilley, D. Biochemistry 1998, 37, 17629-36. 5. Giuliano, K.; Taylor, D. Trends Biotechnol 1998, 16, 135-40. 6. Lehn, J. M. Science 1993, 260, 1762-3. 7. Bier, F. F.; Furste, J. P. Exs 1997, 80, 97-120. 8. Osborne, S. E.; Matsumura, I.; Ellington, A. D. Curr Opin Chem Biol 1997, 1, 5-9. 9. Westhof, E.; Patel, D. J. Curr Opin Struct Biol 1997, 7, 305-9. 10. Patel, D. J.; Suri, A. K.; Jiang, F.; Jiang, L.; Fan, P.; Kumar, R. A.; Nonin, S. J Mol Biol 1997, 272, 645-64. 11. Lin, C. H.; Patel, D. J. Chem Biol 1997, 4, 817-32. 12. Jiang, F.; Kumar, R. A.; Jones, R. A.; Patel, D. J. Nature 1996, 382, 183-6. 13. Jiang, L.; Suri, A. K.; Fiala, R.; Patel, D. J. Chem Biol 1997, 4, 35 -50. 14. Dieckmann, T.; Butcher, S. E.; Sassanfar, M.; Szostak, J. W.; Feigon, J. J Mol Biol 1997, 273, 467-78. 15. Dieckmann, T.; Suzuki, E.; Nakamura, G. K.; Feigon, J. Rna 1996, 2, 628-40. 16. Sassanfar, M.; Szostak, J. W. Nature 1993, 364, 550-3. 17. Huizenga, D. E.; Szostak, J. W. Biochemistry 1995, 34, 656-65. 18. Uphoff, K. W.; Bell, S. D.; Ellington, A. D. Curr Opin Struct Biol 1996, 6, 281-8. 19. Conrad, R. C.; Giver, L.; Tian, Y.; Ellington, A. D. Methods Enzymol 1996, 267, 336-67. 20. Andrus, A.; Cox, S.; Beavers, S.; Parker, A.; Anuskiewicz, J.; Mullah, B. Nucleic Acids Symp Ser 1997, 37, 317-8. 2 1. Scaringe, S. A.; Francklyn, C.; Usman, N. Nucleic Acids Res 1990, 18, 5433-41. 22. Maglott, E. J.; Glick, G. D. Nucleic Acids Res 1998, 26, 1301-8. 23. Wincott, F.; DiRenzo, A.; Shaffer, C.; Grimm, S.; Tracz, D.; Workman, C.; Sweedler, D.; Gonzalez, C.; Scaringe, S.; Usman, N. Nucleic Acids Res 1995, 23, 2677-84.

Any patents or publications mentioned in this specification are indicative of the levels of those skilled in the art to which the invention pertains. Further, these patents and publications are incorporated by reference herein to the same extent as if each individual publication was specifically and individually indicated to be incorporated by reference.

One skilled in the art will readily appreciate that the present invention is well adapted to carry out the objects and obtain the ends and advantages mentioned, as well as those inherent therein. The present examples along with the methods, procedures, treatments, molecules and specific compounds described herein are presently representative of preferred embodiments, are exemplary, and are not intended as limitations on the scope of the invention. Changes therein and other uses will occur to those skilled in the art which are encompassed within the spirit of the invention as defined by the scope of the claims.

What is claimed is:

1. A method of transducing the conformational change of a signaling aptamer upon binding a ligand to a differential signal generated by a reporter molecule comprising the steps of:
contacting the signaling aptamer with the ligand wherein the signaling aptamer binds the ligand; and
detecting the differential signal generated by the reporter molecule resulting from the conformational change of the signaling aptamer upon binding the ligand thereby transducing the conformational change.

2. The method of claim 1, wherein the differential signal comprises an optical signal, an electrochemical signal or an enzymatic signal.

3. The method of claim 2, wherein the optical signal is selected from the group consisting of fluorescence, colorimetric intensity, anisotropy, polarization, lifetime, emission wavelength, and excitation wavelength.

4. The method of claim 1, wherein the signaling aptamer comprises a reporter molecule appended to a nucleic acid binding species (aptamer).

5. The method of claim 4, wherein the reporter molecule is appended to the nucleic acid binding species (aptamer) by covalent coupling or non-covalent coupling.

6. The method of claim 5, wherein the covalent coupling of the reporter molecule to the aptamer occurs during chemical synthesis, during transcription or post-transcriptionally.

7. The method of claim 5, wherein the reporter molecule is a dye.

8. The method of claim 7, wherein the dye is a fluorescent dye.

9. The method of claim 8, wherein the fluorescent dye is selected from the group consisting of acridine and fluorescein.

10. The method of claim 4, wherein the aptamer is selected from the group consisting of RNA, DNA, modified RNA and modified DNA, and wherein the aptamer is not a protein or a biopolymer.

11. The method of claim 1, wherein the ligand is a molecule bound by the signaling aptamer wherein the molecule is not a nucleic acid sequence.

12. The method of claim 1, wherein the ligand is in solution.

13. The method of claim 1, wherein the signaling aptamer is in solution or immobilized on a solid support.

14. The method of claim 13, wherein the signaling aptamer is immobilized on a solid support in parallel wherein the immobilization forms signaling aptamer chips.

15. A method of transducing the conformational change of a signaling aptamer upon binding a ligand to an optical signal generated by a fluorescent dye comprising the steps:
contacting the signaling aptamer with the ligand wherein the signaling aptamer binds the ligand; and
detecting the optical signal generated by the fluorescent dye resulting from the conformational change of the signaling aptamer upon binding the ligand thereby transducing the conformational change.

16. The method of claim 15, wherein the optical signal is selected from the group consisting of fluorescence, colorimetric intensity, anisotropy, polarization, lifetime, emission wavelength, and excitation wavelength.

17. The method of claim 15, wherein the signaling aptamer comprises a fluorescent dye appended to a nucleic acid binding species (aptamer) by covalent coupling of the fluorescent dye to the aptamer.

18. The method of claim 17, wherein the fluorescent dye replaces a nucleic acid residue in the aptamer or is inserted between two nucleic acid residues in the aptamer; wherein the placement does not interfere with the ligand-binding site of the aptamer.

19. The method of claim 17, wherein the fluorescent dye is fluorescein or acridine.

20. The method of claim 17, wherein the aptamer is an anti-adenosine RNA aptamer or an anti-adenosine DNA aptamer.

21. The method of claim 20, wherein the anti-adenosine RNA aptamer is ATP-R-Ac13.

22. The method of claim 20, wherein the anti-adenosine DNA aptamer is DFL7-8.

23. The method of claim 15, wherein the ligand is a molecule bound by the signaling aptamer wherein the molecule is not a nucleic acid sequence.

24. The method of claim 23, wherein the ligand is adenosine.

25. The method of claim 15, wherein the ligand is in solution.

26. The method of claim 15, wherein the signaling aptamer is in solution or immobilized on a solid support.

27. The method of claim 26, wherein the signaling aptamer is immobilized on a solid support in parallel wherein the immobilization forms signaling aptamer chips.

28. A method for quantitating the ligand of claim 15 comprising the steps of:
contacting the signaling aptamer of claim 15 with the ligand wherein the signaling aptamer binds the ligand; and
measuring the increase in the optical signal of claim 15 resulting from the signaling aptamer binding the ligand; wherein the increase in the optical signal positively correlates with the quantity of ligand bound to the signaling aptamer.
